(19) United States
(12) Patent Application Publication
(10) Pub. No.: US 2018/0092737 A1
(43) Pub. Date: Apr. 5, 2018

(54) BREAST TREATMENT DEVICE

(71) Applicant: LifeCell Corporation, Branchburg, NJ (US)

(72) Inventors: Aaron Barere, Hoboken, NJ (US); Antoni Jankiewicz, Branchburg, NJ (US); Nathaniel Bachrach, Clifton, NJ (US)

(21) Appl. No.: 15/722,482

(22) Filed: Oct. 2, 2017

Related U.S. Application Data
(60) Provisional application No. 62/403,344, filed on Oct. 3, 2016.

(51) Int. Cl. A61F 2/12; A61F 2/00; A61F 2/52

(52) U.S. Cl. CPC A61F 2/12; A61F 2/0059; A61F 2230/0063; A61L 2430/04; A61F 2/52

(57) ABSTRACT

The present disclosure provides devices and methods for treating a breast. The system can include a low-density implant, wherein the implant comprises a material that does not allow fluid accumulation within the implant. In addition, the system can include an acellular tissue matrix composition, wherein the low-density implant and acellular tissue matrix are configured to allow implantation of the low-density implant and acellular tissue matrix composition within a breast site.

This application claims priority under 35 USC § 119 to U.S. Provisional Application No. 62/403,344, which was filed on Oct. 3, 2016, and which is herein incorporated by reference in its entirety.

The present disclosure relates generally to devices for improving breast surgeries, including light-weight implants that may be used as temporary or permanent space fillers.

Breast reconstruction post mastectomy has evolved over the last half century due to advances in surgical techniques and technology. Initially, surgeons required the use of tissue expanders post mastectomy to expand the skin due to insufficient remaining tissue. Tissue expanders were inflated to the desired size over a period of weeks or months to create a breast pocket that matched the approximate size and shape of the contralateral breast. Surgeons generally placed these devices in the sub muscular position (i.e., beneath the pectoralis major and serratus muscles), and while aesthetic results were not ideal, this procedure did afford benefits.

Later advances in surgical techniques included the use of the latissimus dorsi muscle. As with prior techniques, the pectoralis major is elevated and a tissue expander is placed beneath it. However, instead of raising the serratus to cover the lower pole of the expander, the latissimus muscle is moved from the back of the patient, and sutured to the edge of the pectoralis to cover the lower pole and form the inframammary fold by attaching the latissimus muscle to the chest wall.

Still later, ALLODERM® (LIFECELL CORPORATION, Branchburg, N.J.), and other acellular tissue matrix products, were added. ALLODERM® can be used to reinforce weakened skin in the lower pole, thereby eliminating the need to mobilize the latissimus dorsi for the same purpose. As a result, surgeons have been able to achieve similar aesthetic results as with the latissimus procedure without the morbidity associated with dissecting and moving that muscle (e.g., donor site pain and complications, and lack of muscle functionality).

In addition, there has been a greater acceptance of skin-sparing and skin and nipple-sparing mastectomies. With such procedures, there is more skin for reconstruction and less of a need for expansion of the breast-skin envelope. As a result, the intended functionality of traditional tissue expanders is often no longer necessary, and surgeons may instead use a device that surrogates for the shape and volume of the tissue removed during the mastectomy. This is well approximated by the size and shape of the proposed final silicone breast implant and, as such, well understood. While one can use a final implant to this end, surgeons face some limitations, including lack of suture tabs, heavy weight, lack of adjustability of size and volume, and cost.

Generally, the breast-skin flaps are not healthy because they have lost the blood supply from the underlying breast tissue removed during mastectomy. Among other things, there is not always a sufficient blood supply to clear infections, which can be especially challenging in the presence of synthetic implants. As a result, surgeons have found that when the silicone implants are placed under well vascularized muscle or rapidly vascularizing acellular dermal matrix (ADM) the incidence of implant loss is reduced.

Placement of the ADM, however, can be challenging. For example, the inferior edge of the ADM defines the inframammary fold where it attaches to the chest wall and reinforces the lower pole of the breast. For less experienced surgeons, or in cases where radical dissection of native tissue occurs, it can be difficult to accurately suture the ADM in the correct position or create the desired shape and curvature. Furthermore, surgeons have discovered that apposition of materials such as ALLODERM® with overlying skin flaps is important for long-term success.

It is possible to achieve good apposition between ADM and tissue using properly expanded tissue expanders or silicone implants. However, use of such devices can create some complications. For example, tissue expanders and silicone implants are relatively heavy, which can cause complications. Accordingly, there is a need for improved devices for breast procedures that can be used in conjunction with various ADMs and in a variety of different surgical procedures.

The present disclosure provides improved devices and methods for performing breast surgeries.

According to one aspect, a breast treatment system is provided. The system can include a low-density implant, wherein the implant comprises a material that does not allow fluid accumulation within the implant. In addition, the system can include an acellular tissue matrix composition, wherein the low-density implant and acellular tissue matrix are configured to allow implantation of the low-density implant and acellular tissue matrix composition within a breast site such that the low-density implant is held in contact with at least a portion of the acellular tissue matrix and holds the acellular tissue matrix in contact with surrounding tissue.

Also provided herein are methods of treating a breast. The methods can include performing a surgical procedure on a breast; implanting a low-density, implant within a space within the breast, wherein the implant comprises a material that does not allow fluid accumulation within the implant; and closing a surgical incision to maintain the low-density implant within the space within the breast. As discussed below, the methods can be used in conjunction with a variety of surgical procedures, can include later removal of the implant, or can further include implantation of a tissue matrix in contact with the implant and surrounding tissue.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIG. 1A is a perspective view of a low-density breast implant, according to one embodiment.

FIG. 1B is a frontal view of the low-density breast implant of FIG. 1A.

FIG. 1C is a side view of the low-density breast implant of FIG. 1A

FIG. 2 is a perspective view for a breast treatment system, including a low-density implant and regenerative tissue matrix, according to various embodiments.

FIG. 3 is a side view of a low-density breast implant and tissue matrix implanted in a subpectoral position, according to various embodiments.

FIG. 4 is a side, cut-away view of a low density breast implant and tissue matrix implanted subcutaneously, according to various embodiments.

FIG. 5A is a perspective view of another configuration for a low-density breast implant, according to certain embodiments.

FIG. 5B is a perspective view of another configuration for a low-density breast implant, according to certain embodiments.

FIG. 5C is a perspective view of another configuration for a low-density breast implant, according to certain embodiments.

FIG. 6 is a side, cut-away view of another configuration for a low-density breast implant, including an outer shell to prevent tissue ingrowth and attachment, according to certain embodiments.

FIG. 7 is a side, cut-away view of another configuration for a low-density breast implant, including an internal structure for adjusting the implant volume, according to certain embodiments.

FIG. 8 is a flow chart showing steps for treating a breast using the low-density implants and/or tissue matrices of the present disclosure.

### DESCRIPTION OF EXEMPLARY EMBODIMENTS

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure relates generally to devices for surgical breast procedures and systems and methods relating to such devices. The devices can be used for tissue augmentation, repair or regeneration of damaged tissue, and/or correction of tissue defects. As such, the devices, systems, and methods discussed herein can be suitable for a wide range of surgical applications such as, for example, aesthetic surgery, breast reconstruction, breast augmentation, breast enhancement, breast reduction, and revisionary breast surgeries.

The devices of the present disclosure include low-density implants that can be used immediately following breast surgery, for example, immediately after skin-sparing mastectomy to maintain and/or augment the space that was occupied by the breast and subcutaneous tissue removed during a breast procedure. The device can facilitate proper placement of materials such as acellular dermal matrix and can specifically create a surface that accurately refelcts the shape of the breast skin envelope prior to a surgery, or a desired post-surgical size and shape, so that the acellular dermal matrix can be placed in the correct position without pleating or similar alteration.

The devices can also provide adequate pressure between the acellular tissue matrix and surrounding tissue (e.g., a skin flap in skin-sparing mastectomy), such that integration of the tissue matrix to the skin flap is improved.

According to one aspect, a breast treatment system is provided. The system can include a low-density implant, wherein the implant comprises a material that does not allow fluid accumulation within the implant. In addition, the system can include an acellular tissue matrix composition, wherein the low-density implant and acellular tissue matrix are configured to allow implantation of the low-density implant and acellular tissue matrix composition within a breast site such that the low-density implant is held in contact with at least a portion of the acellular tissue matrix and holds the acellular tissue matrix in contact with surrounding tissue.

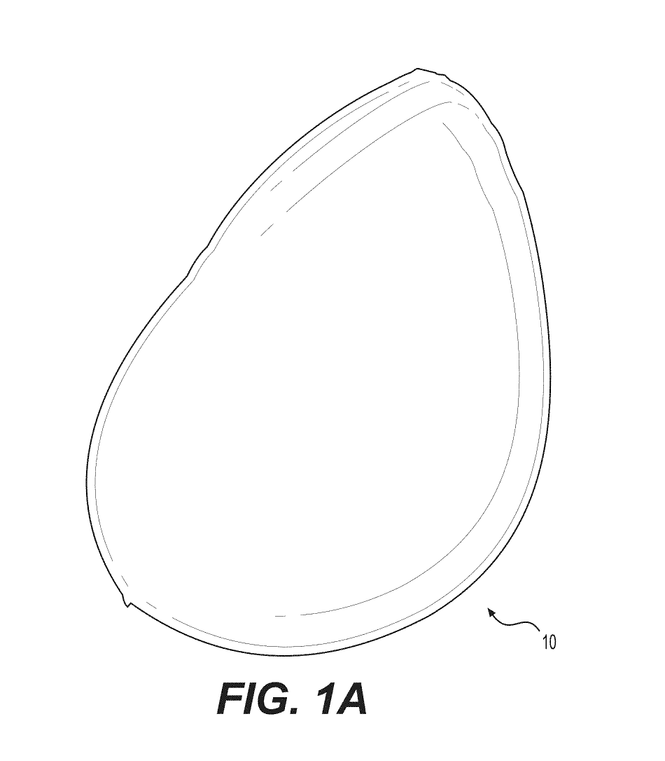

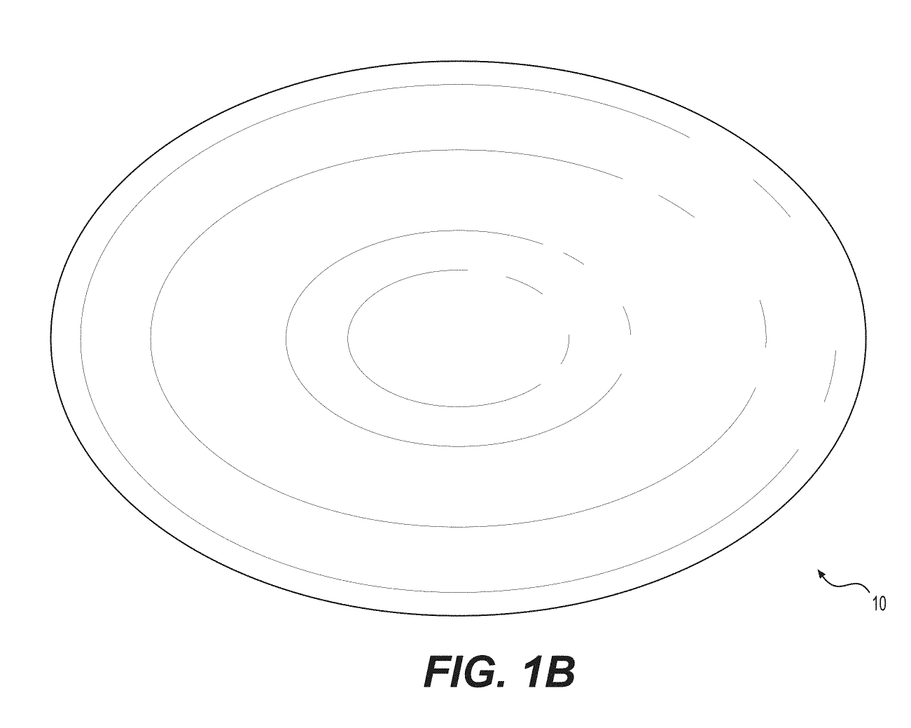

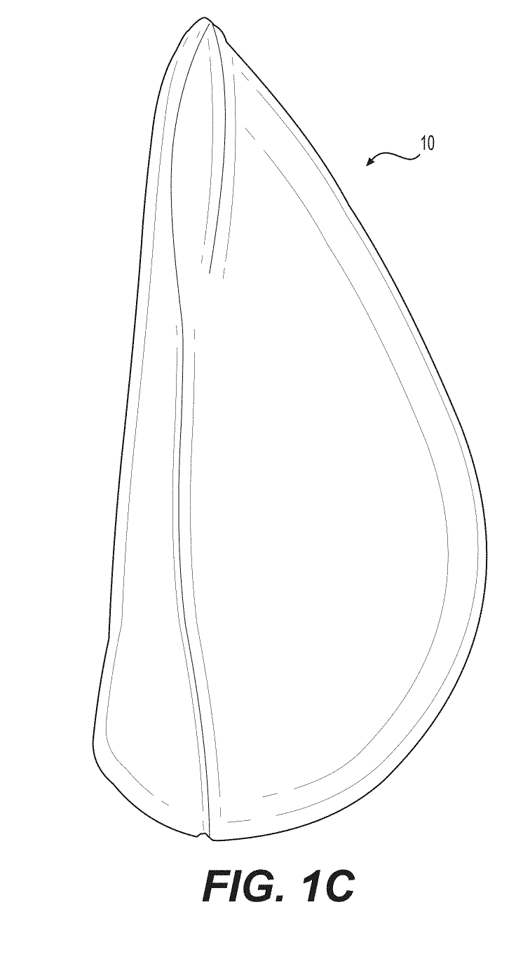

FIGS. 1A-C are perspective, frontal, and side views of a low-density breast implant, respectively, according to one embodiment of the present disclosure. As shown, the implant 10 can have a teardrop shape, which is common for breast implants. However, as described below, the shape of the implant can be varied based on the indication or patient characteristics and can include rounded shapes, irregular shapes, custom-made shapes, or two or more discrete components-all selected based on the specific indication and patient factors.

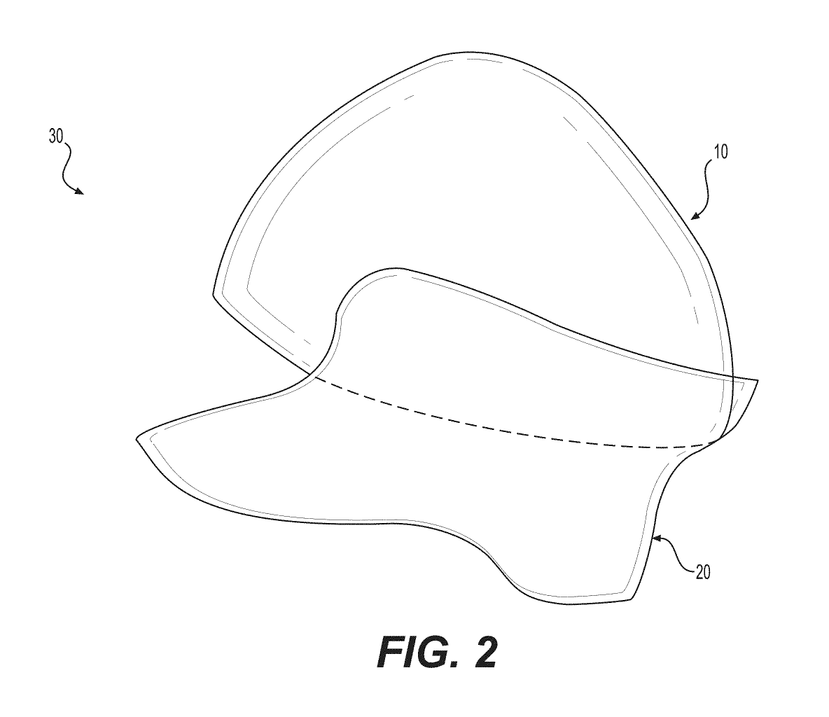

FIG. 2 is a perspective view for a breast treatment system 30, including a low-density implant 10 and regenerative tissue matrix 20, according to various embodiments. As shown, the implant 10 and tissue matrix 20 can be provided as separate components, and when implanted, the tissue matrix 20 can be positioned in apposition to the implant 10. For example, the implant 10 may be positioned within a breast (e.g., in a subpectoral position, subcutaneous position, or any other site), and the tissue matrix 20 can be placed around at least a portion of the implant 10 such that the tissue matrix 20 contacts the implant and is pushed against surrounding tissue, thereby improving blood flow and cellular ingrowth from surrounding tissues.

The implant 10 can be formed of a variety of suitable materials. As noted above, the implant 10 can be formed of a low-density material that does not permit accumulation of fluid within the implant, and can be biocompatible, temperature stable (e.g., does not melt or soften excessively when implanted), and non-biodegradable. The low-density material is selected to provide a lightweight device that will produce little stress on surrounding tissues, thereby preventing surgery failure due to host tissue or implant failure, especially during periods shortly after surgery when surrounding tissues are healing and while tissue is being formed within the regenerative tissue matrix 20.

Furthermore, the implant can be produced from a material that does not permit fluid accumulation to (1) prevent weight increase, (2) prevent ingrowth of cells and tissues, (3) and prevent increased infection risk due to fluid ingress. By preventing ingrowth of cells and tissues, the implant 20 may be removed at a later time (e.g., after healing of tissues and generation of a supporting tissue structure with the tissue matrix 20).

A variety of suitable low-density materials may be selected. For example, in one embodiment, the materials comprise a closed-cell foam. Suitable polymers that may be used to form such foams can include polyurethane, silicone, polyvinylchloride, polytetrafluoroethylene, acrylate-based polymers, and/or polyethylene. In some embodiments, more than 60% of the cells, more than 70% of the cells, more than 80% of the cells, or 90% of the cells can be closed within the closed-cell foam.

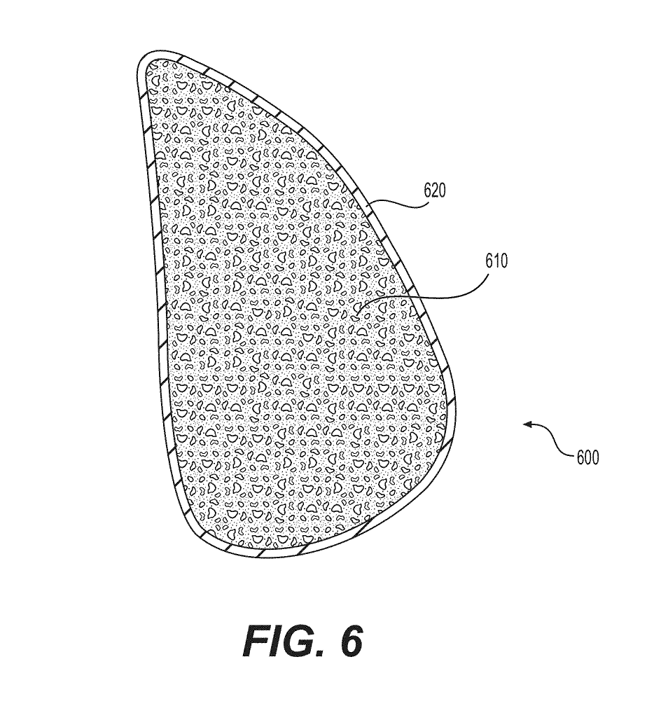

Alternatively, or in addition to including a closed-cell foam, the implant may be formed of an open- or closed-cell foam covered by an outer moisture-impermeable layer to prevent fluid and cell ingress. The outer layer can be biocompatible, temperature stable (e.g., does not melt or soften excessively when implanted), and non-biodegradable. For example, FIG. 6 is a side, cut-away view of another configuration for a low-density breast implant 600 including an outer shell 620 to prevent tissue ingrowth and attachment with an inner core 610. In some embodiments, the material including an outer moisture-impermeable layer can be an integral skin foam and can be created, for example, using an open-mold or closed-mold process. In some embodiments, the material including an outer moisture-impermeable layer can be created using reaction injection molding.

In some embodiments, the low-density material can have a pore size of less than 200 microns, less than 100 microns, or less than 50 microns. In some embodiments, the outer moisture-impermeable layer can have a pore size of less than 200 microns, less than 100 microns, or less than 50 microns. In embodiments with an outer moisture-impermeable layer, the interior of the implant can be formed of a material having a larger pore size than the outer layer.

The low-density breast implant can include a low-density material that is sufficiently compressible according to application-specific requirements. In some embodiments, the density of the low-density material can be chosen to achieve a certain weight or mass for the implant 20 at a given implant size or volume. In embodiments where the entire implant is comprised of a closed-cell foam, the density of the low-density material can be chosen to be low. In embodiments where the implant includes an outer moisture-impermeable layer, the outer layer can be formed of a closed-cell foam or other material of higher density, and the interior of the implant can be formed of a lower density material.

For example, silicone or saline breast implants can range in volume from 100 cm³ to 800 cm³. The masses of silicon or saline breast implants can range from 100 g to 800 g. In accordance with various embodiments, low-density implants as described herein can have masses of between 1% and 10%, 1% and 5%, 1% and 20%, between 10% and 30%, 20% and 30%, 10% and 40%, or between 20% and 50% of the mass of a similarly sized or shaped silicone or saline implant. In accordance with various embodiments, low-density implants as described herein can have masses of less than 50%, 45%, 40%, 35%, 30%, 25%, 20%, 15%, 10%, 5%, or 1% of the mass of a similarly sized or shaped silicone or saline implant. In some embodiments, a surgeon can choose the material composition of a low-density implant based upon patient-specific factors such as the extent or severity of damage to surrounding tissue, breast size or shape, and amount of remaining tissue after an operation. In various embodiments, the density of the low-density material can be between 0.02 g/cm³ and 1.2 g/cm³ or any density corresponding to a percent mass, as listed above, that will allow a desired mass as compared to the mass of a similarly sized saline-filled implant or tissue expander.

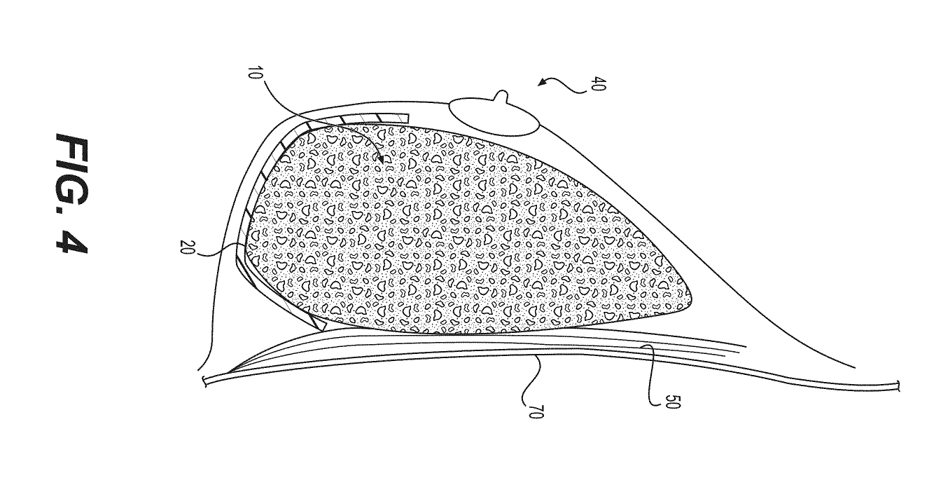

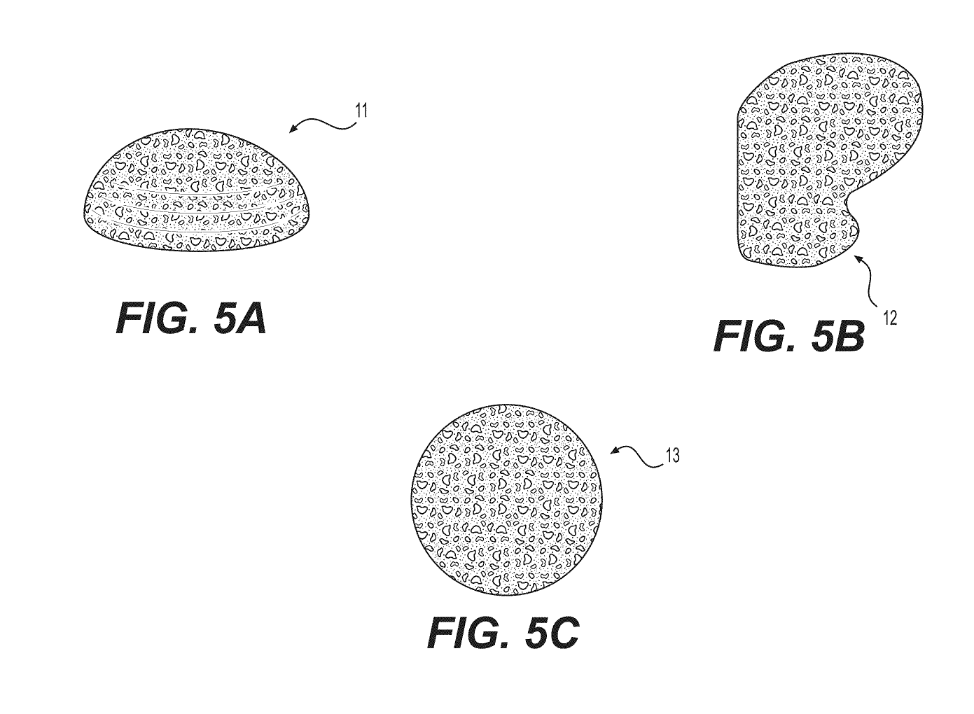

As noted above, the implant 10 can have a variety of shapes, sizes and configurations. For example, as shown in FIGS. 1A-C, the implant can be teardrop shaped, which is common for many breast implants. Alternatively, the implant 10 can have a rounded configuration, as shown in FIG. 5A, which is also common for breast implants. Furthermore, these implants can have a range of suitable sizes, including variations in size (based on width and/or volume) or projection.

In addition, the implants of the present disclosure need not have typical breast implant (teardrop or rounded) shapes. For example, the implants can have other shapes including, for example, irregular shapes (FIG. 5B), spherical shapes (FIG. 5C), ovoid shapes, or custom-made shapes based on patient anatomy or treatment site. For example, a surgeon may select a spherical or custom-made shape for implantation in a lumpectomy site or based on patient-specific factors. In addition, the surgeon may select two or more implants to be implanted next to one another or in different locations. In addition, although described in particular with respect to breast implants, the presently disclosed implants, systems, and methods can be used at other sites where synthetic implants may be used (e.g., gluteal implants).

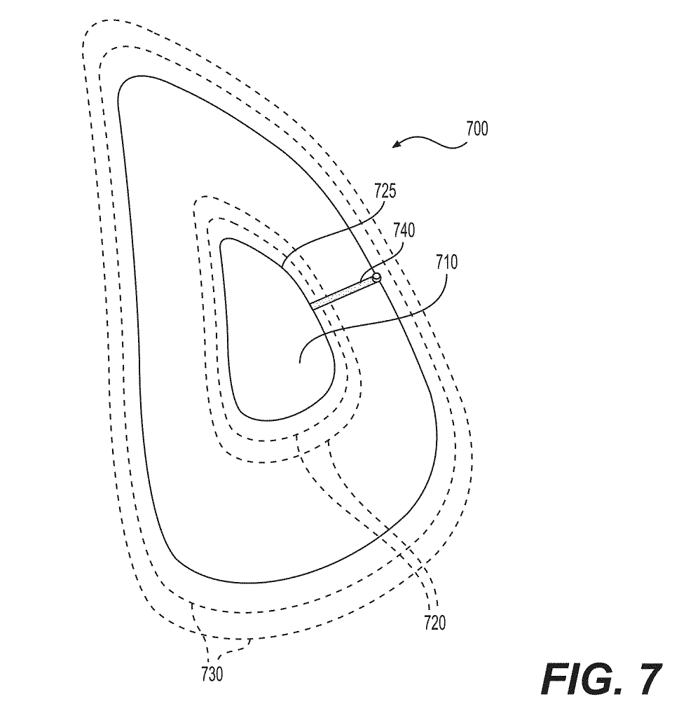

The implants may also have features that permit some change in shape or volume either before or after implantation. For example, FIG. 7 is a side, cut-away view of another configuration for a low-density breast implant 700, including an internal structure 710 for adjusting the implant volume, according to certain embodiments. As shown, the structure 710 can include a hollow chamber or flexible wall 725 that can increase to large sizes 720, thereby increasing the size of the outer wall 730 of the implant 700. The structure 710 may be a hollowing within the implant 700 or may include a bladder with a discrete wall 725.

In various embodiments, the internal structure 710 can be configured to allow control of specific shapes or device attributes. For example, the structure can be designed such that injection of fluid effects a change in a particular implant dimension (e.g., base size, projection, or volume, or some selected combination thereof). For example, in one embodiment, the implant may allow little expansion in one characteristic (e.g., base size), while allowing easy expansion in a direction that increases desired dimension such as projection. Such control may be effected by controlling bladder elastic properties, e.g., by selective cross-linking or control of material thickness.

In order to adjust the implant volume, a surgeon may insert or remove fluids (e.g., saline), gels, or gases, from the internal structure 710. For example, the implant may include a port 740 for receiving a needle or cannula to transfer fluid. Alternatively, the implant can be formed of a material that can receive a narrow-bore needle to inject fluid, but which will be self-sealing.

Also provided herein are methods of treating a breast. The methods can include performing a surgical procedure on a breast; implanting a low-density implant within a space within the breast, wherein the implant comprises a material that does not allow fluid accumulation within the implant; and closing a surgical incision to maintain the low-density implant within the space within the breast. As discussed below, the methods can be used in conjunction with a variety of surgical procedures, can include later removal of the implant, and can further include implantation of a tissue matrix in contact with the implant.

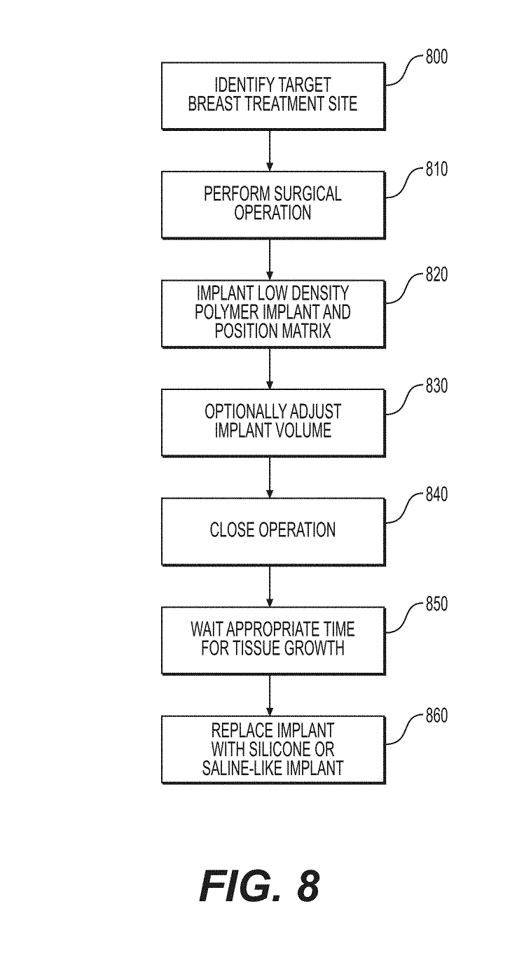

FIG. 8 is a flow chart showing steps for treating a breast using the low-density implants and/or tissue matrices of the present disclosure. As shown, the process starts by selecting a site for treatment (Step 800). The selection will often be based on the need to remove tissue, for example, in surgical oncology, to remove breast tissue during mastectomy, skin-sparing mastectomy, lumpectomy, or any other procedure such as revision breast augmentation, breast augmentation, or mastopexy. It should be noted that as used herein, mastectomy or lumpectomy can include any variations on such procedures, such as radical mastectomy, modified radical mastectomy, or lumpectomy with sentinel node biopsy. In some cases, the site may be selected for augmentation or aesthetic procedures, and the procedure may include simply accessing the surgical site, and/or altering the site appearance.

After the site is selected, the surgical procedure (e.g., mastectomy, lumpectomy, surgical access) is performed (Step 810), and the low-density implant 10 (or variations thereof described above) is implanted (Step 820). At the same time, a tissue matrix is implanted and correctly positioned, for example, by partially wrapping around the implant, and if necessary, anchoring the matrix to surrounding tissues or the implant. In some cases, a tissue matrix may be omitted, and the surgery closed with just the implant in place. In such cases, the surgeon may allow tissue to heal around the implant and consider implantation of the tissue matrix later.

In some cases, a surgeon may adjust the volume of the implant (Step 830) either to achieve desired aesthetic results, achieve proper apposition or placement of the tissue matrix and surrounding tissue, or to stretch tissue a desired amount. The surgeon may then close the incision (Step 840). The implant volume can be adjusted as described with devices illustrated in FIG. 7. It should be noted that the surgeon may elect to adjust the implant volume during surgery or post-operatively, e.g., through percutaneous access to ports 740. Such adjustment may be performed if a change in aesthetic is needed, to further expand tissues, or both.

After closing the incision, the surgeon may wait a period of time (Step 850), during which the patient will heal from the operation, and during which time tissue can form within the tissue matrix. After an appropriate period (e.g., weeks or months), the surgeon can then operate again and remove the implant. If appropriate, the implant can then be replaced by a more traditional breast implant such as a silicone or saline-filled implant, or if needed, a normal tissue expander (Step 860).

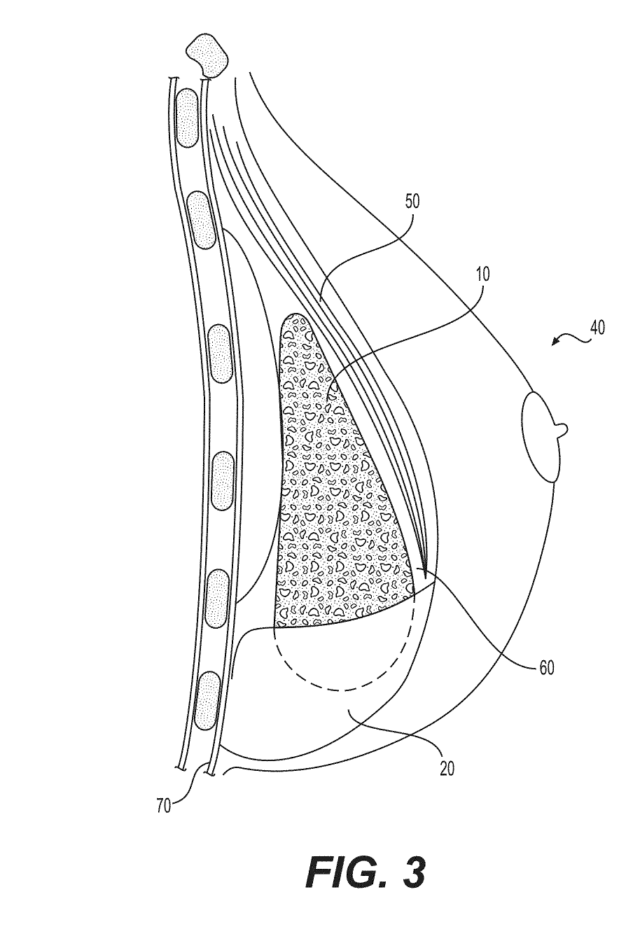

As noted above, the implants and systems described herein can be used in a variety of anatomical sites and in various surgical methods. For example, FIG. 3 is a side view of a low-density breast implant 10 and tissue matrix 20 implanted in a subpectoral position, according to various embodiments. FIG. 4 is a side, cut-away view of a low density breast implant 10 and tissue matrix 20 implanted subcutaneously. In both cases, the implant 10 and tissue matrix 20 are positioned within a breast 40, and one or both may optionally be sutured or attached to surrounding tissues such as the chest wall 70. As such, the devices of the present disclosure may further include tabs or other structures to facilitate suturing or otherwise securing to the body.

As shown in FIG. 3, the implant 10 can be positioned in a space 60 partially beneath a pectoralis muscle 50, or, as with FIG. 4, can be positioned under the skin, but above the pectoralis muscle 50, as may be the case in a lumpectomy or skin-sparing mastectomy. As shown in FIG. 4, the tissue matrix 20 can partially wrap around the implant 20 to assist in supporting the implant and to allow tissue ingrowth to strengthen a desired area of the breast (e.g., the overlying skin and/or inframammary fold), so that the tissue can support a heavy implant, if implanted later.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

1. A method for treating a breast, comprising:
performing a surgical procedure on a breast;
implanting a low-density implant within a space within the breast, wherein the implant comprises a material that does not allow fluid accumulation within the implant; and
closing a surgical incision to maintain the low-density implant within the space within the breast.

2. The method of claim 1, wherein the surgical procedure comprises a skin-sparing mastectomy.

3. The method of claim 1, wherein the surgical procedure comprises at least one of a mastectomy, a lumpectomy, a revision breast augmentation procedure, or a mastopexy.

4. The method of claim 1, wherein the implant comprises a closed-cell foam implant.

5. The method of claim 1, wherein the implant comprises an open-cell foam and an outer layer that is substantially impermeable to fluid flow.

6. The method of claim 1, wherein the implant consists of a closed-cell polyurethane.

7. The method of claim 1, wherein the implant comprises a material selected from a polyurethane, silicone, polyvinylchloride, acrylate-based polymers, or polyethylene.

8. The method of claim 1, further comprising adjusting the size of the implant.

9. The method of claim 8, wherein adjusting the size of the implant comprises adjusting the size of a flexible chamber within the implant.

10-13. (canceled)

14. The method of claim 1, further comprising implanting a sheet of material within the breast adjacent to the low-density implant.

15. The method of claim 14, wherein the sheet of material comprises an acellular tissue matrix.

16. The method of claim 15, wherein the acellular tissue matrix comprises a dermal acellular tissue matrix.

17-18. (canceled)

19. The method of claim 18, further comprising implanting a second breast implant within the breast.

20. The method of claim 19, wherein the second breast implant comprises a silicone-filled or saline-filled implant.

21. The method of claim 1, wherein the low-density implant is a tear-drop shaped implant.

22. The method of claim 1, wherein the low-density implant is a rounded implant.

23. (canceled)

24. A breast treatment system, comprising:
A low-density implant, wherein the implant comprises a material that does not allow fluid accumulation within the implant; and
an acellular tissue matrix composition, wherein the low-density implant and acellular tissue matrix are configured to allow implantation of the low-density implant and acellular tissue matrix composition within a breast site such that the low-density implant is held in contact with at least a portion of the acellular tissue matrix and the low density implant holds the acellular tissue matrix in contact with surrounding tissue.

25. The system of claim 24, wherein the low-density implant comprises an open-cell foam covered with an outer layer that is substantially impermeable to fluid.

26. The system of claim 24, wherein the implant consists of a closed-cell polyurethane.

27. The system of claim 24, wherein the implant comprises a material selected from a polyurethane, silicone, polyvinylchloride, acrylate-based polymers, or polyethylene.

28. The system of claim 24, wherein the implant further comprises a flexible chamber within the implant.

29. The system of claim 24, wherein the chamber comprises a bladder with a flexible wall.

30. (canceled)

31. The system of claim 24, wherein the acellular tissue matrix comprises a human acellular tissue matrix.

32. The system of claim 31, wherein the acellular tissue matrix comprises a dermal acellular tissue matrix.

33. The system of claim 24, wherein the implant is a tear-drop shaped implant.

34. The system of claim 24, wherein the implant is a rounded implant.

35. (canceled)
